(19) United States
(12) Patent Application Publication
(10) Pub. No.: US 2025/0025384 A1
(43) Pub. Date: Jan. 23, 2025

(54) SILICONE-FREE INVERSE EMULSION COMPRISING A C3 OR C4 ALKANEDIOL, GLYCEROL, A NONVOLATILE VEGETABLE OIL, A POLYGLYCERYL POLYRICINOLEATE SURFACTANT AND A VOLATILE ALKANE

(71) Applicant: L'OREAL, Paris (FR)

(72) Inventors: Fabrice SPRINGINSFELD, Chevilly Larue (FR); Valérie DIQUE-MOUTON, Chevilly Larue (FR); Pascal ARNAUD, Chevilly (FR); Florence L'ALLORET, Chevilly Larue (FR)

(73) Assignee: L'OREAL, Paris (FR)

(21) Appl. No.: 18/716,212

(22) PCT Filed: Nov. 18, 2022
(86) PCT No.: PCT/EP2022/082414

§ 371 Date: Jun. 4, 2024

(30) Foreign Application Priority Data
Dec. 6, 2021 (FR) 2113001

(51) Int. Cl. A61K 8/06; A61K 8/31; A61K 8/34; A61K 8/39; A61K 8/92; A61Q 1/02

(52) U.S. Cl. CPC A61K 8/064; A61K 8/31; A61K 8/345; A61K 8/39; A61K 8/922; A61Q 1/02; A61K 2800/34; A61K 2800/43

(57) ABSTRACT

The present patent application relates to a silicone-free composition in the form of a water-in-oil emulsion, comprising, in particular in a physiologically acceptable medium: a) a continuous oily phase containing: i) at least one nonvolatile vegetable oil; and ii) at least one volatile alkane; and b) an aqueous phase dispersed in said oily phase containing: c) at least one polyglyceryl polyricinoleate surfactant; and d) at least one C₃ or C₄ alkanediol; and e) at least glycerol. The invention also relates to a method for coating keratin materials, more particularly for making up and/or caring for keratin materials, such as the skin, characterized in that it comprises the application, to the keratin materials, of a composition as defined above.

### TECHNICAL FIELD

The present invention is targeted at providing, for the field of the care and/or makeup of keratin materials, in particular of the skin, a new composition in the form of an inverse emulsion which is very particularly advantageous with regard to its technical performance qualities and the sensory feelings which it provides the user with when it is applied on these keratin materials and in particular on the skin.

Cosmetic compositions, for example foundations, are commonly used to give the skin an esthetic color, but also to enhance the beauty of irregular skin, by making it possible to hide marks and dyschromias, to reduce the visibility of relief imperfections, such as pores and wrinkles, and to conceal spots and acne marks; in this regard, coverage is one of the main properties sought.

In the cosmetics field, water-in-oil emulsions, also called inverse emulsions, are particularly appreciated by consumers in the field of foundations, sun protection products or moisturizing creams, with regard to their cosmetic properties, in particular with regard to their comfort on application, which is reflected in particular by an absence of or a strong reduction in the feeling of tightness, of drying and/or the tacky and/or greasy effect, in comparison with “direct” oil-in-water emulsions. These same inverse emulsions also make it possible to contain a large amount of fillers and/or pigments and to obtain good homogeneity during application, in comparison with direct emulsions. However, such compositions exhibit the disadvantage of being generally difficult to stabilize.

Consumers are also increasingly looking for cosmetic products formed, in all or in part, of natural constituents or constituents of natural origin, whether for reasons of better skin tolerance or for reasons of environmental protection.

The term “natural compound” is understood to mean a compound which is obtained directly from the earth or the soil, or from plants or animals, via, if appropriate, one or more physical processes, such as, for example, milling, refining, distillation, purification or filtration.

The term “compound of natural origin” is understood to mean a natural compound which has undergone one or more additional chemical or industrial treatments, bringing about modifications which do not affect the essential qualities of this compound, and/or a compound predominantly comprising natural constituents which have or have not undergone transformations. Mention may be made, as nonlimiting example of additional chemical or industrial treatment bringing about modifications which do not affect the essential qualities of a natural compound, of those permitted by the controlling bodies, such as Ecocert (Reference system for biological and ecological cosmetic products, January 2003), or defined in recognized handbooks in the field, such as “Cosmetics and Toiletries Magazine”, 2005, Vol. 120, 9: 10.

The use of nonvolatile vegetable oils in the formulation of cosmetic compositions has been known since antiquity. These oils have emollient and moisturizing properties and make it possible to confer great softness on the skin.

However, nonvolatile vegetable oils, due to their high polarity, can reduce the stability of emulsions. What is more, these oils can give the compositions which include them an excessive greasy and tacky nature, during and after their application, in particular when they are present in a high content. In addition, they can be the source of a shiny effect, which can prove to be detrimental to the desired cosmetic properties, in particular in the context of care or makeup of the skin, such as with foundations.

The abovementioned disadvantages can be all the more pronounced when the cosmetic composition is an inverse emulsion comprising an external fatty phase.

To overcome these drawbacks, it has already been proposed to combine these nonvolatile vegetable oils with silicone oils, in particular volatile ones, so as to reduce their greasy and tacky nature and thus to favor the application of the cosmetic compositions. However, this solution does not meet the expectations of consumers who today are looking for cosmetic products derived from “green chemistry”, namely made up predominantly of natural compounds or compounds of natural origin.

For obvious reasons, taking this expectation of users into account in the development of conventional emulsions raises difficulties with regard in particular to incompatibilities between these “natural” compounds and those conventionally retained, and which are detrimental to the stability of the emulsions.

Thus, the use of polyglyceryl polyricinoleate emulsifying surfactants, which constitute, as described in the patent DE 44 09 569, a favored class of surfactants of natural origin insofar as they result from an esterification reaction of polyglycerol and of acid of vegetable origin, can detrimentally affect the stability of the compositions comprising them, being reflected in particular by problems of coalescence.

To overcome this disadvantage, it has been proposed to introduce high levels of polyols into the aqueous phase of these emulsions, so as to increase the stability of the compositions. U.S. Pat. No. 6,013,255 and WO 2008/055692 describe the use of a polyol in emulsions comprising polyglyceryl polyricinoleate.

However, this solution can give rise to a feeling of tackiness on application or after making up and thus does not always prove to be satisfactory in terms of comfort on application, in particular in terms of the ease on application, such as good slip.

The patent FR 2 933 868 has proposed inverse emulsions comprising at least one volatile linear alkane of natural origin, such as those described in the patent application WO 2007/068371 of Cognis and at least one calcium carbonate.

The patent FR 2 936 154 has also proposed inverse emulsions comprising at least one volatile linear alkane, such as those described in the patent application WO 2007/068371 of Cognis, at least one polyglyceryl polyricinoleate and at least one polyol chosen more particularly from sorbitol in combination with glycerol.

However, these compositions are not entirely satisfactory in terms of stability, in terms of sensory properties, such as the absence of a tacky effect, of good comfort properties, such as ease on application, such as good slip.

The need exists to find novel cosmetic compositions for caring for and/or making up keratin materials, in particular the skin, in the form of a water-in-oil emulsion comprising natural compounds or compounds of natural origin, which are stable, exhibit good sensory makeup properties, good properties of comfort on application, without the abovementioned disadvantages.

The applicant company, during its research studies, has unexpectedly discovered that this objective was achieved by using a silicone-free composition in the form of a water-in-oil emulsion, comprising, in particular in a physiologically acceptable medium:
a) a continuous oily phase containing:
i) at least one nonvolatile vegetable oil; and
ii) at least one volatile alkane; and
b) an aqueous phase dispersed in said oily phase; and
c) at least one polyglyceryl polyricinoleate surfactant; and
d) at least one C₃ or C₄ alkanediol; and
e) at least glycerol.

This discovery forms the basis of the invention.

### SUBJECT MATTERS OF THE INVENTION

Thus, according to one of its aspects, the present invention relates to a silicone-free composition in the form of a water-in-oil emulsion, comprising, in particular in a physiologically acceptable medium:
a) a continuous oily phase containing:
i) at least one nonvolatile vegetable oil; and
ii) at least one volatile alkane; and
b) an aqueous phase dispersed in said oily phase; and
c) at least one polyglyceryl polyricinoleate surfactant; and
d) at least one C₃ or C₄ alkanediol; and
e) at least glycerol.

The invention also relates to a method for coating keratin materials, more particularly for making up and/or caring for keratin materials, such as the skin, characterized in that it comprises the application, to the keratin materials, of a composition as defined above.

### Definitions

In the context of the present invention, the term “keratin material” is understood in particular to mean the skin (body, face, area around the eyes), the lips, the eyelashes and the eyebrows. More particularly, the term “keratin material” is understood to mean the skin.

The term “physiologically acceptable” is understood to mean compatible with the skin and/or its superficial body growths, which exhibits a pleasant color, odor and feel, and which does not cause unacceptable discomfort (stinging or tightness) liable to dissuade the consumer from using this composition.

Within the meaning of the present invention, the term “water-in-oil emulsion”, also referred to as inverse emulsion, is understood to denote any composition constituted of a continuous oily phase in which the aqueous phase is dispersed in the form of droplets, so as to observe a mixture which is macroscopically homogeneous to the naked eye.

The term “silicone” is understood to mean any compound comprising at least one silicon atom and in particular at least one Si—O group.

The term “silicone-free composition” is understood to mean any composition containing less than 1.0% by weight of silicone compound, indeed even less than 0.5% by weight, indeed even less than 0.1% by weight, with respect to the total weight of the composition, indeed even devoid of silicone compound.

### Oily Phase

A cosmetic composition in accordance with the present invention comprises an oily phase containing at least one nonvolatile vegetable oil and at least one volatile alkane.

The term “oil” is understood to mean any fatty substance in the liquid form at ambient temperature (20-25° C.) and at atmospheric pressure.

A composition of the invention can comprise an oily phase in a content varying from 5% to 95%, in particular from 10% to 80%, in particular from 15% to 70% and more particularly from 20% to 65% by weight, with respect to the total weight of the composition.

### a) Vegetable Oils

The oily phase of the composition of the invention comprises at least one nonvolatile vegetable oil.

The term “nonvolatile oil” is understood to mean an oil which remains on the keratin material at ambient temperature and atmospheric pressure for at least several hours and which has in particular a vapor pressure of less than 10⁻³ mmHg (0.13 Pa).

A vegetable oil can be extracted from a plant product either by a single cold pressing (virgin oil) or by hot pressing and refining (refined oil).

The nonvolatile vegetable oils of the invention are oils extracted directly from plants without undergoing any chemical modification.

Mention may be made, as nonvolatile vegetable oil suitable for the invention, of aloe oil, sweet almond oil, peach kernel oil, peanut oil, argan oil, avocado oil, candlenut oil, baobab oil, borage oil, broccoli oil, calendula oil, camelina oil, carrot oil, safflower oil, hemp oil, rapeseed oil, cottonseed oil, coconut oil, cucumber seed oil, wheat germ oil, jojoba oil, shea oil, alfalfa oil, lily oil, macadamia oil, corn oil, meadowfoam oil, St. John's wort oil, millet oil, monoi oil, hazelnut oil, apricot kernel oil, walnut oil, olive oil, evening primrose oil, barley oil, palm oil, passionflower oil, poppy oil, blackcurrant seed oil, kiwi seed oil, grape seed oil, pistachio oil, red kuri squash oil, pumpkin oil, quinoa oil, musk rose oil, sesame oil, rye oil, soybean oil, sunflower oil, castor oil and watermelon oil, and their mixtures.

According to one embodiment, a nonvolatile vegetable oil suitable for the invention can in particular be chosen from macadamia oil, olive oil and their mixtures.

A composition according to the invention can comprise from 0.5% to 50% by weight of nonvolatile vegetable oil(s), with respect to the total weight of the composition, in particular from 1% to 25% by weight of vegetable oil and more particularly from 1% to 10% by weight of nonvolatile vegetable oil, with respect to the total weight of the composition.

### b) Volatile Alkanes

The oily phase of the composition of the invention comprises at least one volatile alkane.

The term “alkane” is understood to mean any compound comprising a saturated, linear or branched, hydrocarbon chain constituted exclusively of carbon atoms and hydrogen atoms.

The term “volatile alkane” suitable for the invention is understood to mean a cosmetic alkane which is capable of evaporating on contact with the skin in less than one hour, at ambient temperature (25° C.) and atmospheric pressure (760 mmHg, that is to say 101 325 Pa), which is liquid at ambient temperature and which has in particular an evaporation rate ranging from 0.01 to 15 mg/cm²/minute, at ambient temperature (25° C.) and atmospheric pressure (760 mmHg).

Mention may be made, by way of example of volatile alkane which can be used in the oily phase of the composition of the invention, of branched alkanes, such as C₈-C₁₆ isoalkanes of petroleum origin (also called isoparaffins), such as isododecane (also called 2,2,4,4,6-pentamethylheptane), isodecane, isohexadecane, and for example the oils sold under the trade names of Isopars® or Permethyls®. Use will more particularly be made of isododecane.

Mention may also be made, by way of example of volatile alkane which can be used in the oily phase of the composition of the invention, of volatile linear C₆-C₁₉ alkanes.

Mention may be made, by way of example of linear alkanes suitable for the invention, of the alkanes described in the patent applications WO 2007/068371 and WO 2008/155059 of Cognis (mixtures of different alkanes differing by at least one carbon). These alkanes are obtained from fatty alcohols, themselves obtained from copra oil or palm oil.

Mention may be made, by way of example of linear alkanes suitable for the invention, of n-hexane (C₆), n-heptane (C₇), n-octane (C₈), n-nonane (C₉), n-decane (C₁₀), n-undecane (C₁₁), n-dodecane (C₁₂), n-tridecane (C₁₃), n-tetradecane (C₁₄) and their mixtures.

Mention may in particular be made of n-dodecane (C₁₂) and n-tetradecane (C₁₄), which are sold by Sasol respectively under the references Parafol 12-97® and Parafol 14-97®, and also their mixtures.

According to one embodiment, a mixture of n-dodecane and n-tetradecane is used. Use may in particular be made of the dodecane/tetradecane mixture in the 85/15 ratio by weight sold by Biosynthis under the reference Vegelight 1214©.

According to yet another embodiment, use is made of a mixture of volatile linear C₉-C₁₂ alkanes with the INCI name: C9-12 Alkane, such as the product sold by Biosynthis under the reference Vegelight Silk©.

Use will more particularly be made of a mixture of n-undecane (C₁₁) and n-tridecane (C₁₃), such as those obtained in examples 1 and 2 of the application WO2008/155059 from Cognis and such as that sold under the trade name Cetiol Ultimate® by BASF.

Preferably, the volatile alkane(s) are present in a concentration ranging from 0.5% to 90% by weight, in particular from 1% to 50% by weight and more particularly from 5% to 40% by weight, with respect to the total weight of the composition.

### c) Additional Nonvolatile Oils

According to a specific embodiment, the oily phase can comprise at least one additional nonvolatile hydrocarbon oil (different from the nonvolatile vegetable oils described above).

The term “hydrocarbon oil” is understood to mean an oil containing mainly carbon and hydrogen atoms and possibly one or more functions chosen from hydroxyl, ester, ether or carboxyl functions.

The term “nonvolatile oil” is understood to mean an oil which remains on the keratin material at ambient temperature and atmospheric pressure for at least several hours and which has in particular a vapor pressure of less than 10⁻³ mmHg (0.13 Pa).

Mention may be made, among the additional nonvolatile hydrocarbon oils which can be used according to the invention, of phytosteryl esters, such as phytosteryl oleate, phytosteryl isostearate and phytosteryl/octyldodecyl lauroyl glutamate (Ajinomoto, Eldew PS203); fatty esters, such as diisopropyl sebacate (Stéarineries Dubois, DUB DIS), isopropyl myristate (Stéarineries Dubois, DUB IPM), isopropyl palmitate (Oleon, Radia 7732); triglycerides consisting of esters of fatty acids and of glycerol, in particular, the fatty acids of which can have chain lengths varying from C₄ to C₃₆ and in particular from C₁₈ to C₃₆, it being possible for these oils to be saturated or unsaturated and linear or branched; these oils can, in particular, be heptanoic or octanoic triglycerides, or also caprylic/capric acid triglycerides, such as those sold by Stéarineries Dubois or those sold under the names Miglyol 810@, 812® and 818® by Dynamit Nobel.

Mention may also be made of squalane, such as the product sold under the trade name Neossance Squalane® by Aprinova.

Mention may also be made of dialkyl ether oils having from 10 to 40 carbon atoms, such as dicaprylyl ether, such as the product sold under the trade name Rofetan OE/MB® by Ecogreen Oleochemicals or the product sold under the trade name Cetiol OE/MB® by BASF.

According to a specific form of the invention, a mixture of squalane and dicaprylyl ether will be used.

Preferably, the additional nonvolatile hydrocarbon oil or oils are present in a concentration of less than or equal to 50% by weight and more particularly from 1% to 25% by weight, with respect to the total weight of the composition.

According to a specific embodiment, the ratio by weight of the total amount of vegetable oil(s) to the total amount of volatile alkane(s) varies from 1/40 to 1/1 and preferably from 1/20 to 1/5.

According to a specific embodiment, the ratio by weight of the total amount of nonvolatile vegetable oil(s) and of additional nonvolatile hydrocarbon oil(s) to the total amount of volatile alkane(s) varies from 1/8 to 1/1 and preferably from 1/4 to 1/1.

### Aqueous Phase

The aqueous phase comprises water and optionally water-soluble or water-miscible ingredients, such as water-soluble solvents.

A water suitable for the invention can be a floral water, such as cornflower water, and/or a mineral water, such as Vittel water, Lucas water or La Roche-Posay water, and/or a thermal water.

A composition of the invention can comprise water in a content varying from 5% to 80%, more particularly from 20% to 65% and more preferably still from 30% to 55% by weight, with respect to the total weight of the composition.

The composition according to the invention contains at least glycerol. The glycerol is present in a concentration ranging from 0.5% to 15% by weight, in particular from 1% to 10% by weight and more particularly from 3% to 8% by weight, with respect to the total weight of the composition.

### Polyglyceryl Polyricinoleate Surfactant

A composition according to the invention comprises at least one polyglyceryl polyricinoleate surfactant.

Within the meaning of the present invention, the term “surfactant” is understood to mean an amphiphilic compound, that is to say a compound exhibiting two parts of different polarities. Generally, one is lipophilic (soluble or dispersible in an oily phase).

The other is hydrophilic (soluble or dispersible in water). Emulsifying surfactants are characterized by the value of their HLB (Hydrophilic Lipophilic Balance), the HLB being the ratio of the hydrophilic part to the lipophilic part in the molecule. The term “HLB” is well known to a person skilled in the art and is described, for example, in “The HLB System. A Time-Saving Guide to Emulsifier Selection” (published by ICI Americas Inc; 1984). For the emulsifying surfactants used in the preparation of water-in-oil emulsions, their HLB value is generally less than or equal to 8 and more particularly ranges from 3 to 8. The value of the HLB can be determined by the Griffin method or the Davies method.

Within the meaning of the invention, the term “polyglyceryl polyricinoleate” denotes an ester resulting from the esterification of one or more polyglycerols with at least one polyricinoleic acid.

A polyglycerol suitable for the invention can be chosen from the compounds of following general formula (I):

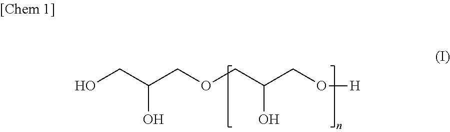

in which n represents an integer of between 1 and 11 and in particular between 1 and 7.

A polyricinoleic acid suitable for the invention can be chosen from the compounds of following general formula (II):

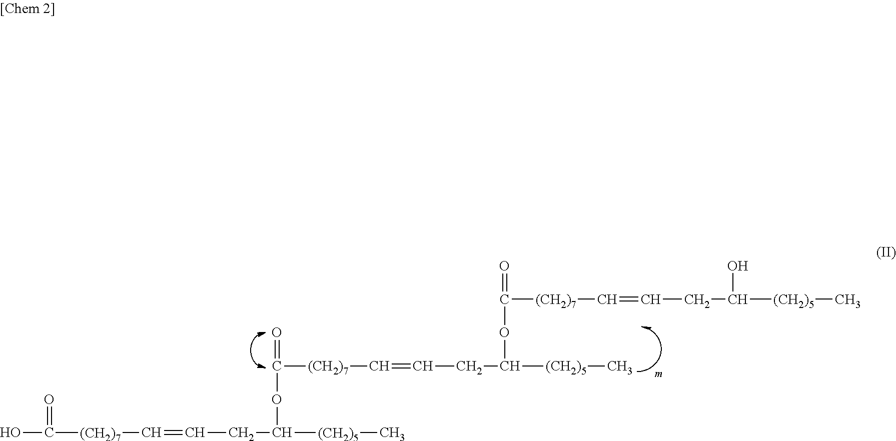

in which m represents an integer of between 0 and 10, in particular between 1 and 8 and more particularly between 1 and 5.

A polyglyceryl polyricinoleate surfactant suitable for the invention can be a total or partial ester.

Preferably, a polyglyceryl polyricinoleate surfactant suitable for the invention is a partial ester.

Within the meaning of the invention, the term “partial ester” is understood to denote a compound in which not all the —OH groups of the polyglycerol units have been esterified by polyricinoleic acid, in other words a polyglyceryl polyricinoleate compound comprising at least one free —OH group on the polyglycerol chain.

By way of example, a polyglyceryl polyricinoleate suitable for the invention can be a compound of following general formula (III):

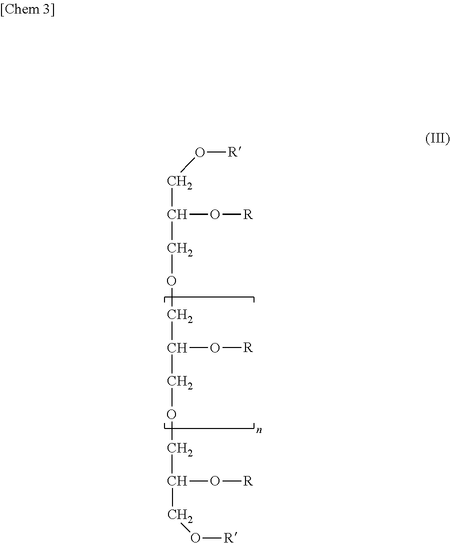

in which R and R′ represent, without distinction, radicals chosen from a hydrogen atom or a polyricinoleate chain, with the proviso that at least one of these R or R′ radicals is a polyricinoleate chain.

Preferably, at least one of the R or R′ groups of the polyglycerol chain is a hydrogen atom.

According to one embodiment, a polyglyceryl polyricinoleate surfactant suitable for the invention can exhibit an HLB (Hydrophilic Lipophilic Balance) of between 3 and 8.

Mention may in particular be made, by way of example of polyglyceryl polyricinoleate surfactant(s) suitable for the invention, of polyglyceryl-3 polyricinoleates, in particular sold by Karlshamns under the name Akoline PGPR® or by Stéarinerie Dubois Fils under the name DUB PGPR or by Dr. Straetmans under the name Dermofeel® or by Croda under the name Crester PR® or also by Sasol under the name Imwitor 600®; the polyglyceryl-5 polyricinoleates sold by Taiyo Kagaku Co. Ltd under the name Sunsoft No. 818R®; the polyglyceryl-6 polyricinoleates sold by Nikko Chemicals Co. Ltd under the name Hexaglyn PR-15® or by Sakamoto Yakuhin Kogyo Co. Ltd under the name S-Face CR-1001®; the polyglyceryl-10 polyricinoleates sold by Nikko Chemicals Co. Ltd under the name Decaglyn PR-20®.

According to one embodiment, mixtures of these compounds can be employed.

More particularly, a polyglyceryl polyricinoleate surfactant suitable for the invention is Polyglyceryl-6 Polyricinoleate.

A composition of the invention preferably comprises from 2% to 10% by weight of polyglyceryl polyricinoleate surfactant, in particular from 3% to 8% by weight and more particularly from 4% to 7% by weight, with respect to the total weight of the composition.

C₃ or C₄ Alkanediol

A composition according to the invention comprises at least one C₃ or C₄ alkanediol.

The term “C₃ or C₄ alkanediol” is understood to mean any saturated, linear or branched, alkane compound comprising a hydrocarbon chain comprising exclusively hydrogen atoms, 3 or 4 carbon atoms and two hydroxyl groups at different positions.

They can be chosen from 2-methyl-1,3-propanediol (INCI name Methylpropanediol), 1,2-propanediol, 1,3-propanediol (INCI name Propanediol), 1,2-butanediol (or 1,2-butylene glycol), 1,4-butanediol (or tetramethylene glycol), 2,3-butanediol (or 2,3-butylene glycol) or 1,3-butanediol (INCI name Butylene Glycol).

Use will more particularly be made of 1,3-propanediol (INCI name Propanediol),

A composition of the invention preferably comprises from 0.1% to 10% by weight of C₃ or C₄ alkanediol(s), in particular from 0.5% to 8% by weight and more particularly from 1% to 5% by weight, with respect to the total weight of the composition.

According to a specific embodiment, the ratio by weight of the total amount of alkanediol(s) to the total amount of glycerol varies from 1/8 to 1/1 and preferably from 1/4 to 1/1.

### Sugar Alcohol

According to a specific embodiment of the invention, the composition according to the invention additionally comprises at least one sugar alcohol.

The term “sugar alcohol” denotes a sugar comprising only hydroxyl functions. These sugar alcohols differ from ketoses and aldoses, which comprise ketone or aldehyde functions. Sugar alcohols are in particular described in the encyclopedia Kirk-Othmer Encyclopedia of Chemical Technology, John Wiley and Sons, “Sugar Alcohols” article, 2005.

Preferably, the sugar alcohols which can be used in the compositions according to the invention correspond to the general formula:

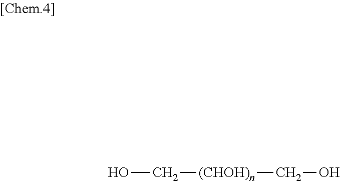

with n an integer ranging from 2 to 5.

Preferentially, the sugar alcohol is chosen from erythritol, sorbitol, threitol, ribitol, arabinitol, allitol, dulcitol, iditol, altritol, lactitol, maltitol, mannitol, xylitol and the mixtures of these compounds.

Particularly preferably, the sugar alcohol is sorbitol (INCI names: Sorbitol or Hydrogenated Starch Hydrolysate).

The composition according to the invention preferably contains from 1% to 15% by weight of sugar alcohol(s), more preferentially still from 2% to 12% by weight, even better still from 4% to 10% by weight, with respect to the total weight of the composition.

Monoester of Saturated and Linear C₇-C₁₄ Carboxylic Acid and of Glycerol

According to a specific embodiment of the invention, the composition according to the invention additionally comprises at least one monoester of saturated and linear C₇-C₁₄ carboxylic acid and of glycerol.

Mention may be made, among monoesters of saturated and linear C₇-C₁₄ carboxylic acid and of glycerol, which can be used in the compositions of the invention, of:
Glyceryl Heptanoate (C₇), such as the product with the trade name Lexgard Natural GH70® (Inolex Inc.);
Glyceryl Caprylate (C₈), such as products with the trade name AEC Glyceryl Caprylate® (A & E Connock Perfumery & Cosmetics); Capmul 708G© (Abitec Corporation); Cremercoor GC 8® (101 Oleo GmbH); Dermosoft GMCY® (Dr. Straetmans GmbH); DUB 8G® (Stéarinerie Dubois Fils); Imwitor 308® and Imwitor 988® (101 Oleo GmbH); Lexgard GMCY MB® (Inolex Inc.); Oristar GCC (Orient Stars LLC); Sunsoft 707® and Sunsoft 700 P-2® (Taiyo Kagaku Company Ltd.); 104528 Symlite G8® (Symrise);
Glyceryl Caprylate (C₈)/Caprate (C₁₀), such as products with the trade name AEC Glyceryl Caprylate/Caprate (A & E Connock Perfumery & Cosmetics Ltd); Cremercoor GC810 (101 Oleo GmbH); Dub 810 G (Stéarinerie Dubois Fils);
Glyceryl Caprate (C₁₀), such as products with the trade name Capmul MCM C-10® (Abitec Corporation); Dermosoft GMC® (Dr. Straetmans GmbH); AEC Glyceryl Caprate® (A & E Connock Perfumery & Cosmetics); Lexgard GMC® (Inolex Inc.); Sunsoft 760 (Taiyo Kagaku Company Ltd);
Glyceryl Laurate (C₁₂), such as the products with the trade name AEC Glyceryl Laurate® (A & E Connock Perfumery & Cosmetics); Colonial Monolaurin® (Colonial Chemical Inc.); DUB LG® (Stéarinerie Dubois Fils); Jeechem MLD® (Jeen International Corporation); Lauricidin (Med-Chem Labs Inc.) Monomuls 90-L 12 (BASF Corporation); Oristar GL (Orient Stars LLC); Pelemol GMLA® (Phoenix Chemical Inc.); Protachem MLD® (Protameen Chemicals); Sunsoft 750® (Taiyo Kagaku Company Ltd); Ultrapure GML® (Ultra Chemical, Inc.)
Glyceryl Myristate (C₁₄), such as products with the trade name AEC Glyceryl Myristate® (A & E Connock Perfumery & Cosmetics); Cithrol GMM (Croda Europe); DUB MG (Stéarinerie Dubois Fils); Nikkol MGM (Nikko Chemicals Co. Ltd); Oristar GMA (Orient Stars LLC); Sunsoft 8002 (Taiyo Kagaku Company Ltd); and
their mixtures.

According to a specific embodiment of the invention, Glyceryl Caprylate will be used.

The composition according to the invention preferably contains from 0.05% to 3% by weight of monoester(s) of saturated and linear C₇-C₁₄ carboxylic acid and of glycerol, more preferentially still from 0.1% to 2% by weight, better still from 0.2% to 1% by weight, with respect to the total weight of the composition.

### Monoalcohol

According to a specific form of the invention, the compositions of the invention additionally comprise at least one linear or branched monoalcohol comprising from 2 to 6 carbon atoms and in particular from 2 to 4 carbon atoms.

The compositions of the invention can comprise one or more monoalcohols.

This monoalcohol can be represented, for example, by the formula RaOH, in which Ra represents a linear or branched alkyl group comprising from 2 to 6 carbon atoms.

Mention may be made, as monoalcohol, of ethanol, isopropanol, propanol, butanol and their mixtures.

According to one embodiment, the compositions of the invention comprise a monoalcohol chosen from isopropanol, ethanol and their mixtures.

According to an advantageous embodiment, the amount of monoalcohol(s) varies from 2% to 15% by weight, preferably from 4% to 10% by weight, with respect to the total weight of said composition.

### Pulverulent Colorants

According to a specific form of the invention, the composition according to the invention additionally comprises at least one pulverulent colorant.

The pulverulent colorants can be chosen from pigments, pearlescent agents and their mixtures.

The term “pigments” is understood to mean white or colored and inorganic or organic particles which are insoluble in an aqueous medium and which are intended to color and/or opacify the resulting composition and/or deposit. These pigments can be white or colored and inorganic and/or organic.

According to a specific embodiment, the pigments used according to the invention are chosen from inorganic pigments.

The term “inorganic pigment” is understood to mean any pigment which satisfies the definition of Ullmann's Encyclopedia in the chapter “Pigments, Inorganic”. Mention may be made, among the inorganic pigments of use in the present invention, of zirconium or cerium oxides, and also zinc, iron (black, yellow or red) or chromium oxides, manganese violet, ultramarine blue, chromium hydrate and ferric blue, titanium dioxide, or metal powders, such as aluminum powder and copper powder. The following inorganic pigments can also be used: Ta₂O₅, Ti₃O₅, Ti₂O₃, TiO, ZrO₂ as a mixture with TiO₂, ZrO₂, Nb₂O₅, CeO₂ or ZnS.

The size of the pigment of use in the context of the present invention is generally greater than 100 nm and can range up to 10 μm, preferably from 200 nm to 5 μm and more preferentially from 300 nm to 1 μm.

According to a specific form of the invention, the pigments exhibit a size characterized by a D[50] of greater than 100 nm and which can range up to 10 μm, preferably from 200 nm to 5 μm and more preferentially from 300 nm to 1 μm.

The sizes are measured by static light scattering using a commercial particle size analyzer of MasterSizer 3000® type from Malvern, making it possible to determine the particle size distribution of all of the particles over a wide range which can extend from 0.01 μm to 1000 μm. The data are processed on the basis of the standard Mie scattering theory. This theory is the most suitable for size distributions ranging from the submicronic to multimicronic; it makes it possible to determine an “effective” particle diameter. This theory is described in particular in the publication by Van de Hulst, H. C., Light Scattering by Small Particles, Chapters 9 and 10, Wiley, New York, 1957.

D[50] represents the maximum size exhibited by 50% by volume of the particles.

According to a specific form of the invention, the inorganic pigment comprises a lipophilic or hydrophobic coating; the latter is preferably present in the fatty phase of the composition according to the invention.

According to a specific embodiment of the invention, the pigments can be coated according to the invention with at least one compound chosen from silicone surface agents; metal soaps; N-acylamino acids or their salts; lecithin and its derivatives; isopropyl triisostearyl titanate; isostearyl sebacate; natural vegetable or animal waxes; polar synthetic waxes; fatty esters; phospholipids; and their mixtures.

According to a preferential embodiment, the pigments can be coated according to the invention with an N-acylamino acid or one of its salts which can comprise an acyl group having from 8 to 22 carbon atoms, such as, for example, a 2-ethylhexanoyl, caproyl, lauroyl, myristoyl, palmitoyl, stearoyl or cocoyl group. The amino acid can, for example, be lysine, glutamic acid or alanine. The salts of these compounds can be the aluminum, magnesium, calcium, zirconium, zinc, sodium or potassium salts. Thus, according to a particularly preferred embodiment, the pigments can be coated with an N-acylamino acid derivative which can in particular be a glutamic acid derivative and/or one of its salts, and more particularly a stearoyl glutamate, such as, for example, aluminum stearoyl glutamate. Mention may be made, as examples of pigments treated with aluminum stearoyl glutamate, of titanium dioxide pigments and black, red and yellow iron oxide pigments sold under the trade name Nai® by Miyoshi Kasei.

According to a preferential embodiment, the pigments can be coated according to the invention with isopropyl triisostearyl titanate. Mention may be made, as examples of pigments treated with isopropyl titanium triisostearate (ITT), of those sold under the trade names BWBO-12® (Iron Oxide CI77499 and Isopropyl Titanium Triisostearate), BWYO-12® (Iron Oxide CI77492 and Isopropyl Titanium Triisostearate) and BWRO-12® (Iron Oxide CI77491 and Isopropyl Titanium Triisostearate) by Kobo.

The pigments which can be used according to the invention can also be organic pigments.

The term “organic pigment” is understood to mean any pigment which satisfies the definition of Ullmann's Encyclopedia in the chapter “Pigments, Organic”. The organic pigment can in particular be chosen from nitroso, nitro, azo, xanthene, quinoline, anthraquinone, phthalocyanine, of metal complex type, isoindolinone, isoindoline, quinacridone, perinone, perylene, diketopyrrolopyrrole, thioindigo, dioxazine, triphenylmethane or quinophthalone compounds.

The organic pigment(s) can be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as are described in the patent FR 2 679 771.

These pigments can also be in the form of composite pigments as are described in the patent EP 1 184 426. These composite pigments can be composed in particular of particles comprising an inorganic core at least partially covered with an organic pigment and at least one binder providing the fixing of the organic pigments to the core.

The pigment can also be a lake. The term “lake” is understood to mean insolubilized dyes adsorbed on insoluble particles, the assembly thus obtained remaining insoluble during use.

The inorganic substrates onto which the dyes are adsorbed are, for example, alumina, silica, calcium sodium borosilicate or calcium aluminum borosilicate, and aluminum.

Mention may be made, among the organic dyes, of cochineal carmine. Mention may also be made of the products known under the following names: D&C Red 21 (CI 45380), D&C Orange 5 (CI 45370), D&C Red 27 (CI 45410), D&C Orange 10 (CI 45425), D&C Red 3 (CI 45430), D&C Red 4 (CI 15510), D&C Red 33 (CI 17200), D&C Yellow 5 (CI 19140), D&C Yellow 6 (CI 15985), D&C Green (CI 61570), D&C Yellow 10 (CI 77002), D&C Green 3 (CI 42053) or D&C Blue 1 (CI 42090).

Mention may be made, by way of an example of a lake, of the product known under the name D&C Red 7 (CI 15 850:1).

Preferably, the composition according to the invention comprises a pulverulent colorant of inorganic pigments type, in particular metal oxides, especially titanium dioxides or iron oxides, which are or are not coated, and their mixtures.

The pearlescent agents can be chosen from white pearlescent pigments, such as mica covered with titanium or with bismuth oxychloride, colored pearlescent pigments, such as titanium oxide-coated mica with iron oxides, titanium oxide-coated mica in particular with ferric blue or chromium oxide, or titanium oxide-coated mica with an organic pigment of the abovementioned type, and also pearlescent pigments based on bismuth oxychloride.

Preferably, said colorant is present in the composition in a content ranging from 0.5% to 30% by weight, preferably from 1% to 25% by weight, more particularly from 3% to 20% by weight, with respect to the total weight of the composition.

### Cosmetic Compositions

The present invention also relates to a cosmetic composition comprising, in a physiologically acceptable medium, a composition as defined above.

The physiologically acceptable medium is generally adapted to the nature of the support onto which the composition has to be applied, and also to the appearance under which the composition has to be packaged.

The compositions according to the invention can additionally comprise additives commonly used in care and/or makeup products, such as:
active agents, such as vitamins, for example vitamins A, E, C or B₃;
sunscreens;
additional colorants;
fillers;
hydrophilic gelling agents;
lipophilic gelling agents;
fragrances;
preservatives;
and their mixtures.

It is a matter of routine operations for a person skilled in the art to adjust the nature and the amount of the additives present in the compositions in accordance with the invention so that the cosmetic properties desired for these are not thereby affected.

### Additional Colorants

A composition according to the invention can additionally comprise at least one additional water-soluble or fat-soluble colorant and preferably in a proportion of at least 0.01% by weight, with respect to the total weight of the composition.

For obvious reasons, this amount is liable to vary significantly with regard to the desired intensity of the color effect and to the color intensity provided by the colorants under consideration, and its adjustment clearly falls within the competence of a person skilled in the art.

The additional colorants suitable for the invention can be fat-soluble.

Within the meaning of the invention, the term “fat-soluble colorant” is understood to mean any natural or synthetic, generally organic, compound which is soluble in an oily phase or solvents miscible with a fatty substance and which is capable of imparting color.

Mention may in particular be made, as fat-soluble dyes suitable for the invention, of synthetic or natural fat-soluble dyes, such as, for example, DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto or curcumin.

The additional colorants suitable for the invention can be water-soluble.

Within the meaning of the invention, the term “water-soluble colorant” is understood to mean any natural or synthetic, generally organic, compound which is soluble in an aqueous phase or water-miscible solvents and which is capable of imparting color.

Mention may in particular be made, as water-soluble dyes suitable for the invention, of synthetic or natural water-soluble dyes, such as, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper chlorophyllin, methylene blue, anthocyanins (enocyanin, black carrot, hibiscus or elder), caramel or riboflavin.

### Fillers

The compositions in accordance with the invention can also comprise at least one filler which makes it possible in particular to confer on them additional properties of improved stability, wear property, coverage and/or mattness.

The term “filler” should be understood as meaning colorless or white solid particles of any shape which are provided in an insoluble form and dispersed in the medium of the composition. These particles, of inorganic or organic nature, make it possible to confer body or firmness on the composition and/or softness and uniformity on the makeup.

The fillers can be inorganic or organic.

Preferably, they will be chosen from natural fillers or fillers of natural origin.

The fillers used in the compositions according to the present invention can be of lamellar, globular, spherical or fibrous forms or of any other form intermediate between these defined forms.

The fillers according to the invention may or may not be surface-coated, and in particular they may be surface-treated with silicones, amino acids, fluorinated derivatives or any other substance which promotes the dispersion and the compatibility of the filler in the composition.

Mention may be made, as examples of inorganic fillers, of talcs, natural or synthetic micas, such as synthetic fluorphlogopites, silica, hollow silica microspheres, kaolin, calcium carbonate, magnesium carbonate, hydroxyapatite, boron nitride, glass or ceramic microcapsules, or composites of silica and of titanium dioxide, such as the TSG® series sold by Nippon Sheet Glass.

Use will more preferentially be made of an inorganic filler chosen from natural or synthetic micas, calcium carbonate and their mixtures.

Mention may be made, as examples of organic fillers, of micronized natural waxes; metal soaps derived from organic carboxylic acids having from 8 to 22 carbon atoms, preferably from 12 to 18 carbon atoms, for example zinc, magnesium or lithium stearate, zinc laurate or magnesium myristate; lauroyl lysine; or cellulose powders, such as that sold by Daito in the Cellulobeads Range®.

Preferably, the filler(s) are present in the composition in a content ranging from 0.5% to 20% by weight, preferably from 1% to 15% by weight, more particularly from 3% to 10% by weight, with respect to the total weight of the composition.

### Gelling Agents

Depending on the viscosity of the composition which it is desired to obtain, one or more gelling agents which are hydrophilic, that is to say soluble or dispersible in water, and/or one or more gelling agents which are lipophilic, that is to say soluble or dispersible in oils, can be incorporated in a composition of the invention.

Preferably, the hydrophilic gelling agents and/or the lipophilic gelling agents will be chosen from natural gelling agents or gelling agents of natural origin.

Mention may in particular be made, as hydrophilic gelling agents, of polysaccharide biopolymers, such as xanthan gum, guar gum, locust bean gum, acacia gum, scleroglucans, chitin and chitosan derivatives, carrageenans, gellans, alginates, celluloses, such as microcrystalline cellulose, carboxymethylcellulose, hydroxymethylcellulose and hydroxypropylcellulose, and their mixtures.

Mention may be made, for example, as lipophilic gelling agents, of modified clays, such as modified magnesium silicate (Bentone Gel VS38® from Rheox), hectorite modified with distearyldimethylammonium chloride (CTFA name: Disteardimonium Hectorite), such as the product sold under the name “Bentone 38 CE®” by Rheox.

According to a particularly preferred embodiment, the composition of the invention in the form of a water-in-oil emulsion comprises, in particular in a physiologically acceptable medium:
a) a continuous oily phase comprising at least:
(i) a mixture of undecane and tridecane; and
(ii) macadamia oil and/or olive oil; and
b) an aqueous phase dispersed in said oily phase; and
c) the surfactant polyglyceryl-6 polyricinoleate; and
d) at least 1,3-propanediol; and
e) at least glycerol.

According to a specific embodiment, said composition additionally comprises f) at least one sugar alcohol and more particularly sorbitol.

According to a specific embodiment, said composition additionally comprises g) at least one monoester of saturated and linear C₇-C₁₄ carboxylic acid and of glycerol and more particularly glyceryl caprylate.

According to a specific embodiment, said composition additionally comprises h) at least one pigment chosen from iron oxides, titanium dioxides and their mixtures; or chosen from iron oxides coated with at least one lipophilic or hydrophobic compound, titanium dioxides coated with at least one lipophilic or hydrophobic compound; and their mixtures.

According to a specific embodiment, said composition additionally comprises i) at least one inorganic filler more particularly chosen from natural or synthetic micas, calcium carbonate and their mixtures.

According to a specific embodiment, said composition additionally comprises j) at least one hydrophilic gelling agent which is natural or of natural origin and/or at least one lipophilic gelling agent which is natural or of natural origin, and more preferentially at least one lipophilic gelling agent chosen from modified clays, and more particularly a hectorite modified with distearyldimethylammonium chloride (INCI name: Disteardimonium Hectorite), such as the product sold under the name “Bentone 38 CE®” by Rheox or the product “Bentone 38VCG” from Elementis.

### Applications

According to one embodiment, a composition of the invention can advantageously be provided in the form of a composition for caring for the skin, in particular of the body or of the face, especially of the face.

According to another embodiment, a composition of the invention can advantageously be provided in the form of a composition for making up keratin materials, in particular the skin of the body or of the face, in particular of the face.

Thus, according to a sub-mode of this embodiment, a composition of the invention can advantageously be provided in the form of a base composition for makeup.

A composition of the invention can advantageously be provided in the form of a foundation.

According to another sub-mode of this embodiment, a composition of the invention can advantageously be provided in the form of a composition for making up the skin and in particular the face. It can thus be an eyeshadow or a face powder.

Such compositions are in particular prepared according to the general knowledge of a person skilled in the art.

### Stability

The term “stable emulsion” is understood to mean, according to the invention, a homogeneous and uniform (water-in-oil) emulsion which does not change in appearance at the macroscopic and microscopic scales at 45° C. for 1 month. The emulsion does not phase separate (separation of the aqueous phase and the fatty phase) and does not release oil.

The stability of a composition of the invention can be evaluated by means of one of the following protocols:

The stability of the compositions in accordance with the invention can be evaluated, for example, by monitoring the change in the size of the globules of the aqueous phase over time.

When the average size of the globules of a composition increases over time, this means that it is subject to coalescence problems, resulting in a phenomenon of heterogeneity and reflecting an instability of the composition.

This monitoring of the change in the size of the globules can be carried out by particle size measurements.

Mention may be made, by way of example of a protocol suitable for the invention, of the following protocol.

Particle size measurements are carried out on 25 ml samples of the compositions to be analyzed, on the very day of their manufacture, and also after storage at 45° C. for 14 days, 30 days and 60 days.

These measurements can be carried out using a MasterSizer 2000® particle size analyzer from Malvern Instruments, making it possible to determine the distribution of particle sizes by diffraction of light. It is then possible to obtain, from the particle size analyzer, the value of the volume-average diameter, denoted D[4,3] and expressed in μm.

The stability of an emulsion can also be evaluated by a visual assessment. For example, the stability of a composition can be evaluated by observation of the extent of the sedimentation taking place for a sample of the composition, after storage at 45° C. for 1 month.

Alternatively, the stability of a composition can be evaluated by observation of a sample of said composition after storage at ambient temperature (ie 25° C.) for 14 days, 30 days and 60 days,

at a magnification of ×10.

Its microscopic appearance must remain close to the initial appearance. In particular, degradation of the emulsion (coarser emulsion background, coalescence reflected by the presence of numerous large drops, modification of the preparation edges, presence of crystals) must not be observed.

Alternatively, the stability of a composition can also be evaluated according to any protocol known to a person skilled in the art.

Throughout the description, including the claims, the expression “comprising a” should be understood as being synonymous with “comprising at least one”, unless otherwise specified.

The expressions “of between . . . and . . . ” and “ranging from . . . to . . . ” should be understood as meaning limits included, unless otherwise specified.

The invention is illustrated in more detail by the examples and figures presented below. Unless otherwise indicated, the amounts shown are expressed as percentage by weight.

### EXAMPLES 1 AND 2 (INVENTION) AND COUNTEREXAMPLES 1, 2, 3, 4 (OUTSIDE THE INVENTION)

Compositions 1 and 2 according to the invention and counterexamples 1, 2, 3 and 4 outside the invention were prepared.

TABLE 1

| | | | Counterex. | Counterex. | Counterex. | Counterex. |
|---|---|---|---|---|---|---|
| Ingredients | Ex. 1 | Ex. 2 | 1 | 2 | 3 | 4 |
| Butylene Glycol | 0 | 2.36 | 0 | 0 | 0 | 0 |
| Disteardimonium | 1.20 | 1.20 | 1.20 | 1.20 | 1.20 | 1.20 |
| Hectorite | | | | | | |
| Alcohol Denat. | 6.32 | 6.32 | 6.32 | 6.32 | 6.32 | 6.32 |
| Aqua | 27.43 | 27.43 | 27.43 | 27.43 | 27.43 | 27.43 |
| Macadamia Ternifolia | 2.01 | 2.01 | 2.01 | 2.01 | 2.01 | 2.01 |
| Seed Oil | | | | | | |
| Glycerin | 5.02 | 5.02 | 7.37 | 0 | 5.02 | 5.02 |
| Sodium Chloride | 0.19 | 0.19 | 0.19 | 0.19 | 0.19 | 0.19 |
| Tocopherol | 0.69 | 0.69 | 0.69 | 0.69 | 0.69 | 0.69 |
| Dicaprylyl Ether | 8.95 | 8.95 | 8.95 | 8.95 | 8.95 | 8.95 |
| Polyglyceryl-2 | 0 | 0 | 0 | 0 | 0 | 6.01 |
| Dipolyhydroxystearate | | | | | | |
| Calcium Carbonate | 5.02 | 5.02 | 5.02 | 5.02 | 5.02 | 5.02 |
| Glyceryl Caprylate | 0.50 | 0.50 | 0.50 | 0.50 | 0.50 | 0.5 |
| Propanediol | 2.36 | 0 | 0 | 7.37 | 0 | 2.36 |
| Undecane (and) | 13.52 | 13.52 | 13.52 | 13.52 | 13.52 | 13.52 |
| Tridecane | | | | | | |
| Polyglyceryl-6 | 6.01 | 6.01 | 6.01 | 6.01 | 6.01 | 0 |
| Polyricinoleate | | | | | | |
| Synthetic | 1.28 | 1.28 | 1.28 | 1.28 | 1.28 | 1.28 |
| Fluorophlogopite | | | | | | |
| Pentylene Glycol | 0 | 0 | 0 | 0 | 2.36 | 0 |
| CI 77891 (and) | 8.71 | 8.71 | 8.71 | 8.71 | 8.71 | 8.71 |
| Disodium Stearoyl | | | | | | |
| Glutamate (and) | | | | | | |
| Aluminum Hydroxide | | | | | | |
| CI 77491 (and) | 0.49 | 0.49 | 0.49 | 0.49 | 0.49 | 0.49 |
| Disodium Stearoyl | | | | | | |
| Glutamate (and) | | | | | | |
| Aluminum Hydroxide | | | | | | |
| CI 77492 (and) | 2.10 | 2.10 | 2.10 | 2.10 | 2.10 | 2.10 |
| Disodium Stearoyl | | | | | | |
| Glutamate (and) | | | | | | |
| Aluminum Hydroxide | | | | | | |
| CI 77499 (and) | 0.20 | 0.20 | 0.20 | 0.20 | 0.20 | 0.20 |
| Disodium Stearoyl | | | | | | |
| Glutamate (and) | | | | | | |
| Aluminum Hydroxide | | | | | | |
| Hydrogenated Starch | 8 | 8 | 8 | 4 | 8 | 8 |
| Hydrolysate | | | | | | |

### Protocol for Preparation of the Compositions

The compositions of the invention are manufactured by preparation of a white base not containing pigments, to which a pigment preparation is added in a second stage.

### a) Preparation of the White Base

The oily phase A1 was introduced into the main beaker, which is placed on a magnetic heating plate (magnetic bar stirring at 200 rev/min, 60° C.).

The hectorite was then introduced (A2) with stirring for 15 minutes until a homogeneous mixture was obtained.

The mixture was subsequently cooled to ambient temperature and kept stirred, then the phases A3 and A4 are added with stirring for 5 minutes. Finally, the phase A5 is introduced while keeping stirred.

The aqueous phase B was prepared by addition of the water, heated to 95° C., to the mixture of the other starting materials of this phase weighed out beforehand.

The emulsion was formed at ambient temperature: the aqueous phase was poured into the fatty phase while gradually increasing the stirring speed up to 4500 rev/min, the formulating being carried out using a Mortiz-type homogenizer.

Stirring was maintained for 10 minutes. Subsequently, the alcohol (phase C) was introduced and the mixture was kept stirred for 5 minutes.

### b) Pigmentation of the White Base

The white base was introduced into a beaker, then the phase D was added slowly with stirring, and stirring was maintained for 20 minutes under vortex. Formulating was carried out using a Rayneri-type homogenizer.

### Protocols and Results of the Tests

### a) Protocol for Evaluation of the Sensory Nature:

Each composition was stored at 25° C. for 24 hours. After cleansing the face of the model, 0.1 ml of the composition to be tested was applied to one cheek and spread with the fingers by massaging in circular motions until the end of the deploying of the foundation.

The evaluation criteria are the playtime (duration of application of the composition to the skin), then the features of tackiness, greasiness and dryness of the skin, 5 minutes after application.

### b) Protocol for Evaluation of the Stability:

The stability of each composition was evaluated after 1 month at 45° C. by observation of its macroscopic appearance and of its microscopic characteristics using a microscope of Leica DM2500 type.

The results of the tests are shown in tables 2 and 3 below.

TABLE 2

| | | Example 2 | | |
|---|---|---|---|---|
| | Example 1 | invention | | |
| | invention | Butylene | Counterexample 1 | Counterexample 2 |
| | Propanediol | Glycol (C₄) + | Glycerin without | Propanediol |
| | (C₃) + Glycerin + | Glycerin + | alkanediol + | without Glycerin + |
| | Polyglyceryl-6 | Polyglyceryl-6 | Polyglyceryl-6 | Polyglyceryl-6 |
| Composition | Polyricinoleate | Polyricinoleate | Polyricinoleate | Polyricinoleate |
| Sensory | Good playtime, | Good playtime, | Short playtime, | Skin dry after |
| properties | Skin slippery | Skin slippery | Skin greasy and | application |
| during and | and non-tacky | and non-tacky | tacky after | |
| after | after application | after application | application | |
| application | | | | |

The results of the tests have shown that examples 1 and 2 of the invention comprising glycerol, a C₃ or C₄ alkanediol (propanediol or butylene glycol) and a polyglyceryl polyricinoleate surfactant, both exhibit a good playtime, and give, after application, skin which is slippery (non-greasy, non-dry) and non-tacky, unlike
counterexample 1 of identical composition but not containing C₃ or C₄ alkanediol;
counterexample 2 of identical composition but not containing glycerol.

TABLE 3

| | | Example 2 | | | |
|---|---|---|---|---|---|
| | Example 1 | invention | Counterex. 1 | Counterex. 3 | |
| | invention | Butylene | Glycerin | Pentylene | Counterex. 4 |
| | Propanediol | Glycol (C₄) + | without | Glycol (C₅) + | Propanediol (C₃) + |
| | (C₃) + Glycerin + | Glycerin + | alkanediol | Glycerin + | Glycerin + |
| | Polyglyceryl-6 | Polyglyceryl-6 | Polyglyceryl-6 | Polyglyceryl-6 | Polyglyceryl-2 |
| Composition | Polyricinoleate | Polyricinoleate | Polyricinoleate | Polyricinoleate | Dipolyhydroxystearate |
| Stability after | Stable | Stable | Unstable, | Unstable, | Unstable, release at |
| 1 month at | | | coalescence | coalescence | the surface |
| 45° C. | | | (appearance | (appearance | |
| | | | of drops of | of drops of | |
| | | | larger size) | larger size) | |

The results of the tests have shown that examples 1 and 2 of the invention comprising glycerin, a C₃ or C₄ alkanediol (propanediol or butylene glycol) and a polyglyceryl polyricinoleate surfactant are stable, unlike
counterexample 1 of identical composition but not containing C₃ or C₄ alkanediol;
counterexample 3 of identical composition but containing a C₅ alkanediol (pentylene glycol);
counterexample 4 of identical composition in which the Polyglyceryl-6 Polyricinoleate surfactant has been replaced by Polyglyceryl-2 Dipolyhydroxystearate.

1. A silicone-free composition in the form of a water-in-oil emulsion, comprising, in particular in a physiologically acceptable medium:
a) a continuous oily phase containing:
i) at least one nonvolatile vegetable oil; and
ii) at least one volatile alkane; and
b) an aqueous phase dispersed in said oily phase:
c) at least one polyglyceryl polyricinoleate surfactant; and
d) at least one C₃ or C₄ alkanediol; and
e) at least glycerol.

2. The composition as claimed in claim 1, comprising an oily phase in a content varying from 5% to 95%, in particular from 10% to 80%, in particular from 15% to 70% and more particularly from 20% to 65% by weight, with respect to the total weight of the composition.

3. The composition as claimed in claim 1 or 2, in which the nonvolatile vegetable oil is chosen from macadamia oil, olive oil and their mixtures.

4. The composition as claimed in any one of the preceding claims, comprising from 0.5% to 50% by weight of nonvolatile vegetable oil(s), in particular ranging from 1% to 25% by weight and more particularly ranging from 1% to 10% by weight, with respect to the total weight of the composition.

5. The composition as claimed in any one of the preceding claims, where the volatile alkane is chosen from branched alkanes, such as C₈-C₁₆ isoalkanes of petroleum origin, in particular chosen from isododecane, isodecane or isohexadecane and more particularly isododecane.

6. The composition as claimed in any one of claims 1 to 4, where the volatile alkane is chosen from volatile linear C₆-C₁₉ alkanes, in particular from n-hexane (C₆), n-heptane (C₇), n-octane (C₈), n-nonane (C₉), n-decane (C₁₀), n-undecane (C₁₁), n-dodecane (C₁₂), n-tridecane (C₁₃), n-tetradecane (C₁₄) and their mixtures.

7. The composition as claimed in claim 6, where the volatile linear alkane is a mixture of volatile linear C₉-C₁₂ alkanes with the INCI name: C9-12 Alkane.

8. The composition as claimed in claim 6, where the volatile linear alkane is a mixture of n-undecane (C₁₁) and n-tridecane (C₁₃).

9. The composition as claimed in any one of the preceding claims, where the volatile alkane(s) are present in a concentration ranging from 0.5% to 90% by weight, in particular from 1% to 50% by weight and more particularly from 5% to 40% by weight, with respect to the total weight of the composition.

10. The composition as claimed in any one of the preceding claims, where the oily phase furthermore comprises at least one additional nonvolatile hydrocarbon oil.

11. The composition as claimed in claim 10, where the additional nonvolatile hydrocarbon oil or oils are present in a concentration of less than or equal to 50% by weight and more particularly ranging from 1% to 25% by weight, with respect to the total weight of the composition.

12. The composition as claimed in any one of the preceding claims, where the ratio by weight of the total amount of nonvolatile vegetable oil(s) to the total amount of volatile alkane(s) varies from 1/40 to 1/1 and preferably from 1/20 to 1/5.

13. The composition as claimed in any one of the preceding claims, the ratio by weight of the total amount of nonvolatile vegetable oil(s) and of nonvolatile hydrocarbon oil(s) to the total amount of volatile alkane(s) varies from 1/8 to 1/1 and preferably from 1/4 to 1/1.

14. The composition as claimed in any one of the preceding claims, comprising water in a content varying from 5% to 80% by weight, more particularly from 20% to 65% by weight and more preferably still from 30% to 55% by weight, with respect to the total weight of the composition.

15. The composition as claimed in any one of the preceding claims, where the glycerol is present in a concentration ranging from 0.5% to 15% by weight, in particular from 1% to 10% by weight and more particularly from 3% to 8% by weight, with respect to the total weight of the composition.

16. The composition as claimed in any one of the preceding claims, where the polyglyceryl polyricinoleate surfactant is Polyglyceryl-6 Polyricinoleate.

17. The composition as claimed in any one of the preceding claims, comprising from 2% to 10% by weight of polyglyceryl polyricinoleate surfactant, in particular from 3% to 8% by weight and more particularly from 4% to 7% by weight, with respect to the total weight of the composition.

18. The composition as claimed in any one of the preceding claims, where the C₃ or C₄ alkanediol is chosen from 2-methyl-1,3-propanediol, 1,2-propanediol, 1,3-propanediol, 1,2-butanediol, 1,4-butanediol, 2,3-butanediol or 1,3-butanediol.

19. The composition as claimed in any one of the preceding claims, where the C₃ or C₄ alkanediol is 1,3-propanediol.

20. The composition as claimed in any one of the preceding claims, where the C₃ or C₄ alkanediol or alkanediol(s) are present at a concentration ranging from 0.1% to 10% by weight, in particular from 0.5% to 8% by weight and more particularly from 1% to 5% by weight, with respect to the total weight of the composition.

21. The composition as claimed in any one of the preceding claims, where the ratio by weight of the total amount of alkanediol(s) to the total amount of glycerol varies from 1/8 to 1/1 and preferably from 1/4 to 1/1.

22. The composition as claimed in any one of the preceding claims, additionally comprising at least one sugar alcohol.

23. The composition as claimed in claim 22, where the sugar alcohol is chosen from erythritol, sorbitol, threitol, ribitol, arabinitol, allitol, dulcitol, iditol, altritol, lactitol, maltitol, mannitol, xylitol and their mixtures, and more particularly sorbitol.

24. The composition as claimed in claim 22 or 23, containing from 1% to 15% by weight of sugar alcohol(s), more preferentially still from 2% to 12% by weight, even better still from 4% to 10% by weight, with respect to the total weight of the composition.

25. The composition as claimed in any one of the preceding claims, additionally comprising at least one monoester of saturated and linear C₇-C₁₄ carboxylic acid and of glycerol.

26. The composition as claimed in claim 25, where the monoester of saturated and linear C₇-C₁₄ carboxylic acid and of glycerol is chosen from:
Glyceryl Heptanoate (C₇);
Glyceryl Caprylate (C₈);
Glyceryl Caprylate (C₈)/Caprate (C₁₀);
Glyceryl Caprate (C₁₀);
Glyceryl Laurate (C₁₂);
Glyceryl Myristate (C₁₄);
their mixtures, and more particularly is Glyceryl Caprylate.

27. The composition as claimed in claim 25 or 26, containing from 0.05% to 3% by weight of monoester of saturated and linear C₇-C₁₄ carboxylic acid and of glycerol, more preferentially still from 0.1% to 2% by weight, better still from 0.2% to 1% by weight, with respect to the total weight of the composition.

28. The composition as claimed in any one of the preceding claims, additionally comprising at least one linear or branched monoalcohol comprising from 2 to 6 carbon atoms and especially from 2 to 4 carbon atoms, in particular chosen from ethanol, isopropanol, propanol, butanol and their mixtures.

29. The composition as claimed in claim 28, where the amount of monoalcohol(s) varies from 2% to 15% by weight, preferably from 4% to 10% by weight, with respect to the total weight of said composition.

30. The composition as claimed in any one of the preceding claims, additionally comprising at least one pulverulent colorant, preferably chosen from pigments, pearlescent agents and their mixtures; and in particular an inorganic pigment chosen from metal oxides, and more particularly titanium dioxides, iron oxides, which are or are not coated, and their mixtures.

31. The composition as claimed in any one of the preceding claims, additionally comprising at least one filler, preferably at least one inorganic filler, more particularly chosen from natural or synthetic micas, calcium carbonate and their mixtures.

32. The composition as claimed in any one of the preceding claims, additionally comprising at least one hydrophilic gelling agent and/or at least one lipophilic gelling agent, said gelling agents preferably being natural or of natural origin.

33. The composition as claimed in claim 32, comprising at least one lipophilic gelling agent chosen from modified clays and more particularly a hectorite modified by distearyldimethylammonium chloride with the INCI name: Disteardimonium Hectorite.

34. A method for coating keratin materials, more particularly for making up and/or caring for keratin materials, such as the skin, characterized in that it comprises the application, to the keratin materials, of a composition as defined in any one of claims 1 to 33.
